(19) United States
(12) Patent Application Publication
(10) Pub. No.: US 2009/0237615 A1
(43) Pub. Date: Sep. 24, 2009

(54) Correction of Surgically-Induced Astigmatism During Intraocular Lens Implants

(76) Inventors: Kamal K. Das, Arlington, TX (US); Xiaoxiao Zhang, Fort Worth, TX (US); Mutlu Karakelle, Fort Worth, TX (US); Xin Hong, Arlington, TX (US)

Correspondence Address: ALCON, IP LEGAL, TB4-8, 6201 SOUTH FREEWAY, FORT WORTH, TX, 76134, US

(21) Appl. No.: 12/417,290

(22) Filed: Apr. 2, 2009

Related U.S. Application Data
(63) Continuation of application No. 11/479,233, filed on Jun. 30, 2006.

(51) Int. Cl. A61B 3/10; A61F 2/14

(52) U.S. Cl. 351/246; 351/177

(57) ABSTRACT

In one aspect, the present invention provides a method of designing an ocular implant (e.g., an IOL), which comprises establishing corneal topography of a patient's eye, e.g., by performing one or more wavefront aberration measurements of the eye, prior to an ocular surgery. The method further includes ascertaining an astigmatic aberration of the cornea that is expected to be induced by the surgery and determining a toricity of a surface of an ocular implant, which is intended for implantation in the patient's eye, so as to enable the implant to compensate for the surgically-induced aberration.

### BACKGROUND

This application claims priority from, and is a continuation of U.S. patent application Ser. No. 11/479,223 filed on Jun. 30, 2006.

The present invention relates generally to methods for designing ophthalmic lenses, and more particularly to methods for customization of intraocular lenses (IOLs) based on individual visual needs of patients.

Intraocular lenses are routinely implanted in patients' eyes during cataract surgery to replace the natural crystalline lens. During the surgery, a small incision is made in the patient's cornea through which instruments can be inserted into the eye to remove the natural lens and introduce an IOL. The incision is typically sufficiently small such that it subsequently heals without a need for sutures. However, the incision, though healed, can induce post-operative corneal aberrations including astigmatism, or modify pre-existing corneal aberrations including astigmatism. Such surgically-induced astigmatism can vary from one patient to another. The corneal astigmatic aberrations can arise due to the curvature of the cornea being unequal at different orientations around the eye's optical axis. Such astigmatism can lead to different magnifications along the two principal meridians, resulting in blurred vision.

Although IOLs having toric surfaces are known that can provide astigmatic correction, traditionally, surgically-induced aberrations are not taken into account in selecting an IOL for implantation in a patient's eye. Hence, an IOL that may be the most suitable one for a patient based on pre-operative measurements of the patient's vision, may not perform as expected due to surgically-induced aberrations.

Accordingly, there is a need for improved methods for designing ocular implants, and in particular ophthalmic lenses, such as IOLs.

### SUMMARY

In one aspect, the present invention provides a method of designing an ocular implant (e.g., an IOL), that comprises establishing a corneal topography of a patient's eye, e.g., by performing one or more wavefront aberration measurements of the eye, prior to an ocular surgery. The method further includes ascertaining one or more aberrations, including astigmatic aberration of the cornea that is expected to be induced by the surgery, and determining a toricity of a surface of an ocular implant, which is intended for implantation in the patient's eye, so as to enable the implant to compensate for the surgically-induced aberration(s).

In a related aspect, the astigmatic aberration induced by the surgery can be determined by modeling the aberration based on the incision type and by employing, e.g., a vector analysis technique.

In another aspect, the ocular surgery comprises a cataract surgery during which an incision is made in the cornea through which instruments can be inserted to remove the natural lens and introduce an intraocular lens. The incision can induce one or more corneal aberrations including astigmatism and/or modify one or more pre-existing aberrations including astigmatism.

In another aspect, in the above method, subsequent to determining the desired toricity, an ocular implant that includes at least one optical surface exhibiting that toricity can be fabricated. By way of example, in some cases, an optical blank having at least one optical surface can be provided, and that surface can be shaped so as to exhibit a desired toricity. In many cases, the optical blank includes two opposed optical surfaces that are shaped such that the resulting optic would provide a requisite optical power as well as compensation for the astigmatic aberration of the patient's eye. The optical blank can be formed of a variety of different materials, such as soft acrylic polymers, hydrogel, polymethylmethacrylate, polysulfone, polystyrene, cellulose, acetate butyrate or other biocompatible polymeric materials having a requisite index of refraction.

In a related aspect, an optic having a surface with a desired degree of toricity can be fabricated by ablating an optical surface of a blank, e.g., by irradiating the surface with ultraviolet radiation. For example, the radiation from an excimer laser (e.g., one operating in a range of about 193 to about 532 nm) can be directed to the surface, e.g., through an appropriate mask, so as to differentially ablate the surface in a manner that would generate a desired toric shape.

In another aspect, a Fast Tool Servo (FTS) machining technique can be employed to shape at least one surface of an optical blank as a desired toric profile. In some cases, the FTS technique can be employed to fabricate optical pins, which can then be used to form a toric IOL.

In another aspect, a method of designing an intraocular lens is disclosed, which includes determining, prior to an ocular surgical operation, a corneal topography of a patient's eye. Aberrations (e.g., astigmatic aberration) of the cornea, including one or more aberrations expected to be induced by the surgery, can then be determined by employing the corneal topography. This is followed by computing a toricity for a surface of an ocular implant adapted to provide compensation for the aberration(s) (e.g., astigmatic aberration) upon implantation in the patient's eye.

In a related aspect, the determination of the corneal topography comprises performing one or more wavefront measurements. Further, the step of determining one or more aberrations (e.g., an astigmatic aberration) comprises modeling one or more aberrations expected to be induced by the surgery and combining those aberration(s) with a pre-existing corneal aberration, if present.

Further understanding of the invention can be obtained by reference to the following detailed description in conjunction with the associated drawings, which are discussed briefly below.

### BRIEF DESCRIPTION OF THE DRAWINGS

FIG. 1 is a flow chart depicting various steps in an exemplary method of the invention for designing an ocular implant;

FIG. 2 is a schematic perspective view of an optical blank;

FIG. 3 is a schematic cross-sectional view of a toric IOL formed by shaping the anterior and posterior surfaces of the optical blank of FIG. 2; and

FIG. 4 schematically shows a diamond blade of an FTS system cutting a selected profile in a substrate.

### DETAILED DESCRIPTION

The present invention generally relates to methods for designing an ocular implant, e.g., an intraocular lens (IOL), for surgical implantation in a patient's eye by taking into account ocular aberrations that can be induced during surgery, e.g., due to incision of the cornea. While the embodiments discussed below are generally directed to methods of designing an IOL for implantation in a patient's eye, the teachings of the invention can be equally applied to other ocular implants, such as intercorneal implants. Further, the term intraocular lens and its abbreviation “IOL” are used herein interchangeably to describe lenses that can be implanted into the interior of an eye to either replace the eye's natural crystalline lens or to otherwise augment vision regardless of whether or not the natural lens is removed.

During a cataract surgery, a small incision is made in the cornea, e.g., by utilizing a diamond blade. An instrument is then inserted through the corneal incision to cut a portion of the anterior lens capsule, typically in a circular fashion, to provide access to the opacified natural lens. An ultrasound or a laser probe is then employed to break up the lens, and the resulting lens fragments are aspirated. A foldable IOL can then be inserted in the capsular bag, e.g., by employing an injector. Once inside the eye, the IOL unfolds to replace the natural lens. The corneal incision is typically sufficiently small such that it heals without the need for sutures. However, in many cases, the incision—though healed—can induce corneal aberrations including astigmatism or modify pre-existing corneal aberrations including astigmatism. In the following embodiments, methods of designing an IOL are disclosed that allow the IOL to compensate for such surgically-induced corneal astigmatism, e.g., on a patient-by-patient basis. In some embodiments, the design methods allow customizing an IOL for a patient based on predicted surgically induced aberrations including astigmatism for that patient.

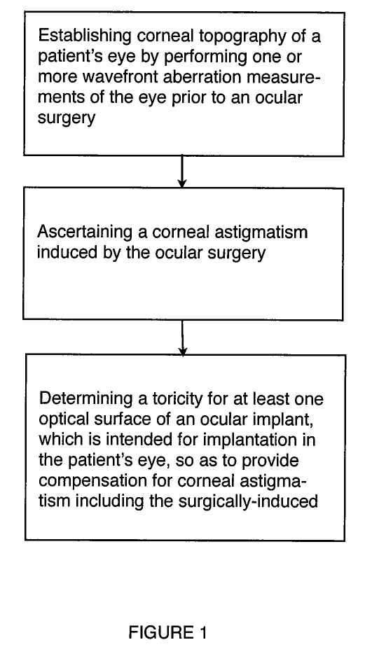

With reference to a flow chart of FIG. 1, in one exemplary embodiment, in an initial step 1, the corneal topography of a patient's eye is established, e.g., by performing one or more corneal elevation map measurements of the eye using a videokeratographer (e.g., one marketed by Humphrey Instruments, San Landro, Calif.) prior to an ocular surgery. By way of example, an article entitled “Optical Aberration of Intraocular Lenses Measured in Vivo And In Vitro,” authored by Barbero and Marcos and published in Journal of Optical Society of America A, vol. 20, pp 1841-1851 (2003), herein incorporated by reference, teaches methods for performing such wavefront aberrations measurements. By way of example, a corneal elevation map can be obtained by a videokeratographer. The elevation height data and their partial derivatives can be inputted to an optical design software (e.g., Zemax software marketed by Focus Software of Tuscon, Ariz.) to obtain the corneal wave aberrations by performing ray tracing.

Referring again to the flow chart of FIG. 1, in a subsequent step 2, an astigmatic aberration of the cornea induced by the surgical incision can be ascertained. By way of example, such an astigmatic aberration can be modeled, e.g., by employing a vector analysis method. Such a vector analysis method models the astigmatic aberration as a vector whose length signifies the aberration amount and whose angle (e.g., relative to a reference axis of a coordinate system in which the vector is represented) signifies twice the cylindrical axis angle of the aberration. Accordingly, the corneal astigmatic aberration prior to the surgical incision can be expressed as a vector, and the astigmatic aberration induced by the surgical incision can be expressed as another vector. Adding these two vectors together, e.g., by employing vector summation rules, can yield a resultant vector, which provides the resultant astigmatic aberration including its amount and its cylindrical axis angle. Further details regarding the vector analysis method can be found, e.g., in the following publications, which are herein incorporated by reference: “Power Vector Analysis of the Optical Outcome of Refractive Surgery,” by Thibos and Horner published in Journal of Cataract Refractive Surgery, vol. 27, pp 80-85 (2001); and “Astigmatic Analysis by the Alpins Method,” by Alpins published in Journal of Cataract Refractive Surgery, vol. 27, pp 29-49 (2001).

Typically, a cataract surgical incision can induce an astigmatism in a range of about ½ D to about 1 D. In some cases, such a surgically-induced astigmatism can modify a pre-existing astigmatism, e.g., worsen or ameliorate the pre-existing astigmatism. Modeling of the effect of the corneal incision in introducing or modifying astigmatic aberrations of the eye can take into account the incision type. By way of example, the effects of a temporal, a superior corneal incision, sub-conjunctival or other corneal incisions (e.g., a 3-mm incision) can be modeled. In many embodiments, other factors that can affect the surgically induced astigmatism (SIA), such as suturing method, presence of suture, incision type, the type of operation and incision width can be also taken into account when modeling SIA. By way of example, these factors are discussed in the following publication, which is herein incorporated by reference: “Optimal Incision Sites to Obtain Astigmatic-Free Cornea After Cataract Surgery With 3.2 mm Sutureless Incision,” by Matsusmoto et al. published in JCRS of Materials Science Letters 27, pp. 1841-1851 (2003).

Subsequently, a toricity for at least one optical surface of an ocular implant (e.g., an IOL) can be determined so as to enable the implant to provide compensation for the corneal astigmatism, including the modeled surgically-induced contribution. By way of example, a model eye having a cornea exhibiting the corneal astigmatic aberration of the patient, including the modeled surgically-induced contribution, can be established. A desired toricity for compensating the astigmatic aberration can then be determined by incorporating a hypothetical ocular implant (e.g., an IOL) in the model eye and varying a toricity of at least one of the implant's surfaces so as to optimize the optical performance of the model eye. In many embodiments, in establishing the model eye for a particular patient, not only the astigmatic aberrations, but also other visual defects of that patient (e.g., myopia, hyperopia) are taken into account.

In some embodiments, the optical performance of the implant can be evaluated by calculating a modulation transfer function (MTF) at the retinal plane of the model eye. As known in the art, an MTF provides a quantitative measure of image contrast exhibited by an optical system, e.g., a model eye incorporating an implant. More specifically, the MTF of an imaging system can be defined as a ratio of a contrast associated with an image of an object formed by the optical system relative to a contrast associated with the object. The human visual system utilizes most spatial frequencies resolvable by neural sampling. Thus, in some embodiments, the MTF values ranging from low (e.g., 10 line pairs (lp)/mm, corresponding to about 20/200 visual acuity) to high (e.g., 100 lp/mm, corresponding to about 20/20 visual acuity) can be averaged to obtain a measure of the optical performance of an implanted IOL. In some embodiments, the toricity of the surface can be varied until a maximal optical performance is obtained.

In some embodiments, the determined toricity of the surface can be mathematically defined, e.g., as a toric surface that can be represented as follows in an XYZ coordinate system (the positive Z-axis is assumed to be the optical axis):

Y²+[X²+(Z−rₕ)²]±2(rₕ−rᵥ)\sqrt{[X²+(Z−rₕ)²]}=rᵥ²−(rₕ−rᵥ)² Eq. (1)

where, rᵥ is the radius of the circle and rₕ is the radius of the outer vertex of the toroid.

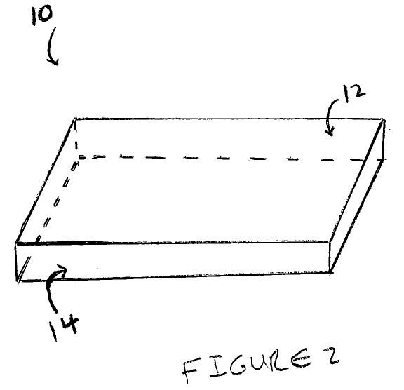

Once a desired toricity is established, an IOL having an optical surface exhibiting that toricity can be fabricated by utilizing a variety of techniques. For example, with reference to FIG. 2, an optical blank 10, formed of a suitable material (such as soft acrylic polymers, hydrogel, polymethylmethacrylate, polysulfone, polystyrene, cellulose, acetate butyrate or other biocompatible polymeric materials having a requisite index of refraction) and having an anterior optical surface 12 and an opposed posterior optical surface 14 can be provided. The anterior and posterior optical surfaces can be shaped, e.g., in a manner discussed below, so as to generate an optic exhibiting a desired optical power (e.g., a power in a range of about −15 D to about 50 D, preferably, in a range of about 6 D to about 34 D). Further, the anterior optic (or the posterior optic) can be shaped so as to compensate for the astigmatic aberration of the cornea of a patient for which the IOL is intended.

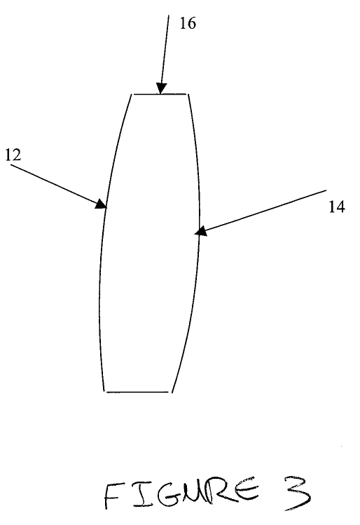

FIG. 3 schematically depicts a cross-sectional view of an IOL 16 obtained by shaping the anterior and posterior surfaces of the optical blank 10. More particularly, in this embodiment, the anterior surface 12 is shaped to have a generally convex profile with a selected degree of toricity adapted to compensate for the astigmatic aberrations of a patient's eye for which the IOL is intended, including a predicted surgically-induced astigmatism. The posterior surface, in turn, is shaped to have a substantially flat profile. By way of example, the anterior surface can exhibit a surface profile defined by the above Equation (1).

In some other embodiments, the surfaces of the optical blank 10 can be shaped by utilizing an ablative laser beam. By way of example, an excimer laser, e.g., an argon-fluoride laser operating at a wavelength of 193 nm, can generate the laser beam. For example, in some cases, a mask having different transparencies at different portions thereof can be disposed between the laser beam and an optical surface of the blank so as to provide differential ablation of different surface portions so as to impart a desired shape to that surface. For example, at least one optical surface of the blank can be shaped so as to have a desired degree of toricity. Further details regarding the use of such ablation methods for fabricating IOLs can be found in U.S. Pat. No. 4,842,782, which is herein incorporated by reference.

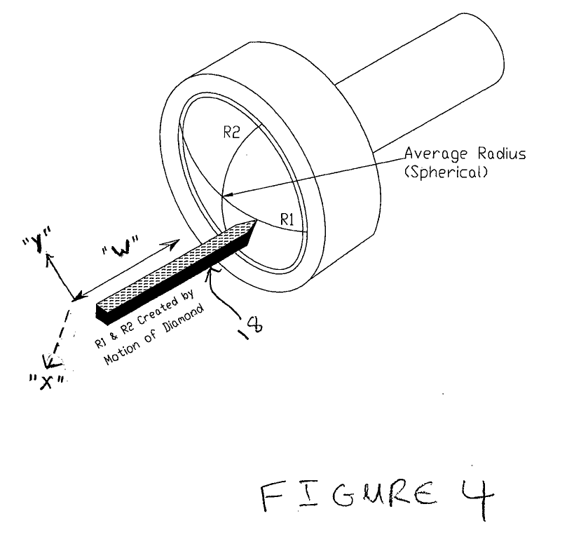

In some embodiments, a machining method, herein referred to as Fast Tool Servo (FTS), is employed for imparting a toric profile to at least one surface of an optical blank. As shown schematically in FIG. 4, the FTS machining method uses a diamond blade 18 that can be made to move along three axes (e.g., ‘X’ and ‘Y’ axes as well as ‘W’ axis that orthogonal to the X-Y plane). More particularly, the diamond blade, under the control of a cutting program, can be made to move along the W direction in a controlled fashion—and typically at a fast rate—while concurrently conducting a two-axis motion (X and Y axes) in a plane perpendicular to the W direction. The combined motions of the blade can result in cutting a desired profile into a substrate's surface.

In some embodiments, the anterior and/or posterior surfaces of an optical blank, such as the above blank 10, can be shaped by employing the FTS machining method. For example, an optical blank formed of a soft acrylic material (cross-linked copolymer of 2-phenylethyl acrylate and 2-phenyl methacrylate) commonly known as Acrysof can be mounted in an FTS system such that a surface thereof faces the system's diamond blade. The motion of the blade can be programmed so as to cut a desired profile, e.g., a toric profile, into the blank's surface. In alternative embodiments, the FTS method can be employed to form optical pins, which can, in turn, be utilized to form the IOL from a desired material. Once the cylindrical axis of the toric profile is defined, it can be marked with axis mark on an optical pin or a lens. Then, when forming a haptic, it can be formed to be aligned with the cylindrical axis mark.

The above methods of designing an IOL advantageously allow custom-making an IOL for an individual patient. For example, prior to performing a cataract surgery on a patient, the patient's corneal topography can be determined, e.g., by utilizing wavefront aberration measurements. By way of example, an ophthalmologist (or other qualified personnel) can perform these measurements. These measurements can then be transmitted to an IOL design and manufacturing facility, which can employ them, together with a predicted surgically-induced astigmatism, to model an IOL suitable for the patient. An IOL can then be fabricated for that patient, which compensates for the astigmatic aberrations, and also corrects other vision defects of that patient.

Those having ordinary skill in the art will appreciate that various changes can be made to the above embodiments without departing from the scope of the invention.

1. A method of designing an ocular implant, comprising
Establishing the topography of a cornea of a patient's eye by performing one or more wavefront aberration measurements of the eye prior to an ocular surgery, ascertaining one or more aberrations, including an astigmatic aberration, of said cornea induced by the surgery, and
determining a toricity for a surface of an ocular implant so as to enable the implant to provide compensation for said one or more surgically-induced aberrations.

2. The method of claim 1, wherein the step of ascertaining the one or more surgically-induced aberrations comprises modeling one or more aberrations caused by said ocular surgery.

3. The method of claim 2, wherein the step of modeling the one or more surgically-induced aberrations comprises employing a vector analysis method.

4. The method of claim 2, further comprising utilizing said wavefront aberration measurements to determine a pre-operative corneal astigmatic aberration.

5. The method of claim 1, further comprising providing an optical blank having at least one optical surface, and shaping said optical surface so as to exhibit said toricity.

6. The method of claim 5, wherein the step of shaping the optical surface further comprises utilizing a Fast Tool Servo (FTS) machining technique.

7. The method of claim 1, further comprising providing an optical blank having at least one ablatable optical surface, and ablating said optical surface so as to generate a toric surface profile characterized by said toricity.

8. The method of claim 7, wherein ablating the optical surface further comprises irradiating the surface with ablating laser energy.

9. The method of claim 8, wherein said laser energy corresponds to laser wavelengths in a range of about 193 to about 532 nm.

10. The method of claim 1, wherein said optical implant comprises an intraocular lens.

11. The method of claim 1, wherein said optical implant comprises a corneal implant.

12. The method of claim 1, wherein said optical implant is formed of a biocompatible material.

13. The method of claim 12, wherein said biocompatible material comprises any of soft acrylic polymers, hydrogel, polymethylmethacrylate, polysulfone, polystyrene, cellulose, and acetate butyrate.

14. A method of designing an intra-ocular lens, comprising
prior to an ocular surgical operation, determining a pre-operative topography of a corneal surface of a patient's eye,
determining one or more aberrations of the cornea including one or more aberrations to be induced by the surgery by employing said corneal topography, and
computing a toricity for a surface of an ocular implant adapted to provide compensation for said one or more aberrations upon implantation in the patient's eye.

15. The method of claim 14, wherein the step of determining the corneal topography comprises performing one or more wavefront aberration measurements.

16. The method of claim 14, wherein said step of determining said one or more aberrations of the cornea comprises modeling the aberration(s) to be induced by the surgery.

17. The method of claim 16, wherein said one or more aberrations comprises astigmatic aberration.

18. The method of claim 17, wherein said step of determining an astigmatic aberration of the cornea comprises combining said modeled aberration with a pre-existing corneal astigmatic aberration.

19. The method of claim 14, wherein said ocular surgery comprises cataract surgery.

20. The method of claim 14, wherein said ocular implant comprises an IOL.
